(19) United States
(12) Patent Application Publication
(10) Pub. No.: US 2008/0132954 A1
(43) Pub. Date: Jun. 5, 2008

(54) Spinal stabilisation implant

(75) Inventors: Lali Sekhon, Reno, NV (US); Stephan J. Duplessis, Calgary (CA); R. John Hurlbert, Calgary (CA)

Correspondence Address: Howard M. Ellis;SIMPSON & SIMPSON, PLLC, 5555 Main Street, Williamsville, NY, 14221, US

(73) Assignee: KINETIC SPINE TECHNOLOGIES, INC., Calgary (CA)

(21) Appl. No.: 11/978,871

(22) Filed: Oct. 30, 2007

Related U.S. Application Data
(63) Continuation of application No. PCT/CA2006/000678, filed on May 2, 2006.
(60) Provisional application No. 60/594,731, filed on May 2, 2005.

(51) Int. Cl. A61B 17/58; A61B 17/56

(52) U.S. Cl. 606/280; 606/246; 606/300

(57) ABSTRACT

A spine stabilization implant is provided for at least two adjacent vertebrae, the implant comprising at least two anchor plates for being secured to the vertebrae and a resilient member extending there-between to simulate a natural ligament. In one embodiment the anchor plates or staples are provided in pairs so as to engage opposite lateral masses of each vertebrae and, thereby, provide bi-lateral stabilization for the spine. In another embodiment, the pairs of anchor plates include a connector extending over the spinous process. In another embodiment, the pairs of anchor plates include one or more connectors to form an artificial spinous process and lamina.

### CROSS REFERENCE TO PRIOR APPLICATIONS

The present application is a Continuation of PCT application no. PCT/CA2006/000678, filed May 2, 2006, which claims priority from U.S. application No. 60/594,731, filed May 2, 2005. The entire disclosures of these applications are incorporated herein by reference.

### FIELD OF THE INVENTION

The present invention relates generally to the field of joint implants and, more particularly, to an implant for use in the stabilisation of spinal elements such as facet joints or other spinal ligaments. More specifically, the invention relates to implants for stabilizing cervical vertebrae of the spine.sadf

### DESCRIPTION OF THE PRIOR ART

The spine is a complicated structure comprised of various anatomical components, which, while being extremely flexible, provides structure and stability for the body. The spine is made up of vertebrae, each having a ventral body of a generally cylindrical shape. Opposed surfaces of adjacent vertebral bodies are connected together and separated by intervertebral discs (or “discs”), comprised of a fibrocartilaginous material. The vertebral bodies are also connected to each other by a complex arrangement of ligaments acting together to limit excessive movement and to provide stability. Vertebrae also include thick lateral portions referred to as lateral masses. Each lateral mass includes facets on the superior and inferior ends thereof. The superior facets of one vertebra are adapted to engage the inferior facets of the next superiorly adjacent vertebra. The engagement of the facets is referred to as a facet joint.

A stable spine is important for preventing incapacitating pain, progressive deformity and/or neurological compromise. Current methods for surgical management of ligamentous insufficiency in the spine involve removal of facet joint capsules and arthrodesis of the joint. In such cases, and in particular in treating instability of the lower cervical spine, it is common to utilize screws extending through the lateral mass of adjacent vertebrae. One of the complications involved in such procedure comprises injury to the spinal nerves during insertion of the lateral mass screws. In addition, with these prior art methods, reconstruction of the facet joint capsule is impossible. Removal of the facet joint eliminates motion at the segment of the spine where the facet joint capsule has been removed, and can lead to accelerated degeneration of adjacent structures.

### SUMMARY OF THE INVENTION

The present invention, in one aspect, provides an implant that obviates or mitigates at least some deficiencies in prior art methods.

In general terms, the invention provides, in one aspect, a spinal stabilization implant having three main components: two staples (or anchor plates) positioned superiorly and inferiorly on the spine, each being secured, respectively, to two adjacent vertebrae; and a resilient synthetic ligament extending there-between. The staples are secured to the spinal structure by screws, pins, bolts and other similar means. Implants as described herein are preferably provided in pairs on laterally opposite sides of the spine. The implants serve to provide resistance to inter-vertebral movement such as during flexion.

In one aspect, the implants described herein are suited for reconstruction of facet joint ligaments and, in such case, the respective staples are secured to lateral masses of vertebrae.

In another aspect, the implants described herein are suited for securing to spinous processes for interspinous and/or supraspinous ligamentous reconstruction.

In another aspect, the implants are adapted to comprise an artificial spinous process and lamina for use as a prosthesis.

Thus, in one aspect, the invention provides a spinal stabilization implant for attaching to two adjacent vertebrae, the vertebrae having one or more bony structures, the implant comprising:
a pair of first spaced apart anchor plates for securing to a first of the vertebrae;
a pair of second spaced apart anchor plates for securing to a second of the vertebrae;
each of the pairs of anchor plates generally being co-planar;
the first and second anchor plates including one or more fastener apertures for receiving fasteners to engage the bony structures of the vertebrae;
each of the pairs of anchor plates being connected to a generally planar fin, the fin being generally perpendicular to the plane containing the respective pairs of anchor plates and wherein the fin includes a first, anchor plate connecting end and an oppositely directed second, free end;
the fins being connected to a resilient member extending there-between.

In another aspect, the invention provides an implant as defined above and wherein the first and second anchor plates are provided in pairs so as to straddle opposite sides of the vertebrae, wherein the implant comprises a pair of first anchor plates are securing to the first vertebra and a pair of second anchor plates for securing to the second vertebra.

In yet another aspect, the invention provides a spinal stabilization prosthetic implant for attaching to two adjacent vertebrae, the vertebrae having one or more bony structures, the implant comprising:
a first anchor plate for securing to a first of the vertebrae;
a second anchor plate for securing to a second of the vertebrae;
the first and second anchor plates including one or more fastener apertures for receiving fasteners to engage the bony structures of the vertebrae;
a resilient member extending between the first and second anchor plates allowing elastic relative movement between the anchor plates.

In another aspect, the above prosthetic implant comprises spacer arms extending between each of the pair of anchor plates and the respective fin thereby connecting the fin to the respective anchor plates.

In yet another aspect, the invention provides a kit for a spinal stabilization implant for attaching to two adjacent vertebrae, the kit comprising:
first and second anchor plate for securing to the vertebrae;
one or more fastening means to fasten the anchor plates to the vertebrae;
at least one resilient member for connecting the first and second anchor plates.

### BRIEF DESCRIPTION OF THE DRAWINGS

Various objects, features and attendant advantages of the present invention will become more fully appreciated and better understood when considered in conjunction with the accompanying drawings, in which like reference characters designate the same or similar parts throughout the several views.

FIG. 1(a) is a top (superior) view of a lateral mass staple according to an embodiment of the invention.

FIG. 1(b) is a side elevation of the staple of FIG. 1(a).

FIG. 2(a) shows a bottom (inferior) view of the staple of FIG. 1(a).

FIG. 2(b) shows a front elevation of the staple of FIG. 1(a).

FIG. 3 is a perspective view of lateral mass staples according to an embodiment of the invention when implanted.

FIG. 4 is a perspective view of an embodiment of the invention when implanted and when the spine is in extension.

FIG. 5 is a perspective view of an embodiment of the invention when implanted and when the spine is in flexion.

FIGS. 6a-6c show plan views of alternate embodiments of the lateral mass staples of the invention.

FIG. 7 is a plan view of an alternate embodiment of the present invention.

FIG. 8(a) is an outer side elevation of a right side portion of a spinous process staple according to an embodiment of the invention.

FIG. 8(b) is an outer side elevation of a left side portion of a spinous process staple according to an embodiment of the invention.

FIG. 8(c) is an inner side elevation of the staples of FIGS. 8(a) or 8(b).

FIG. 8(d) is a side view of a spine wherein the spinous process staples are attached.

FIG. 8(e) is a perspective view of the spinous process staple according to an embodiment of the invention.

FIG. 9a is a posterior elevation of a spine segment illustrating two adjacent vertebrae.

FIG. 9b is a side elevation of the spine segment of FIG. 9a.

FIG. 10a is a plan view of an artificial spinous process according to another aspect of the invention.

FIG. 10b is a perspective side elevation of the device of FIG. 10a.

FIGS. 11a to 11c illustrate the device of FIG. 10a when in use.

### DETAILED DESCRIPTION OF THE INVENTION

In order that the invention may be more fully understood, it will now be described, by way of example, with reference to the accompanying drawings which illustrate embodiments of the present invention.

In the description and drawings herein, and unless noted otherwise, when discussing anatomical plans of view, it will be understood that the terms “front” and “back” shall be used to refer to the front and back in the coronal or frontal plane. The terms “left” and “right” shall be used to refer to left and right in the sagittal or lateral plane. The terms “up” and “down” shall be used to refer to up and down in the axial transverse. It will be understood that a reference to “medial” shall refer towards the midline of a body. It will be understood that a reference to “lateral” shall refer to away from the midline of a body. It will be understood that a reference to “inferior” shall refer to lower, below or down and “superior” shall refer to upper, above or up. It will be further understood that a reference to “anterior” shall refer to front and “posterior” shall refer to the rear or back.

The present invention provides an implant for use in ligamentous reconstruction of joints undergoing or experiencing ligamentous insufficiency. A preferred embodiment of the present invention provides an implant for use in ligamentous reconstruction of joints within the spine undergoing or experiencing ligamentous insufficiency, such as facet or other joints therein. The embodiments of the present invention may also be used to secure ligamentous material to normal or artificial laminae, pedicles, lateral masses, or other regions of the vertebrae. The embodiments of the present invention may also be used to reconstruct joints including spinal joints such as, for example, facet joints or facet joint capsules. While it will be understood that the invention may be used in a variety of joints, including spinal joints in general, a preferred embodiment of the invention is the use of the present invention in facet joints or facet joint capsules collectively referred to as “facet joints” undergoing or experiencing ligamentous insufficiency.

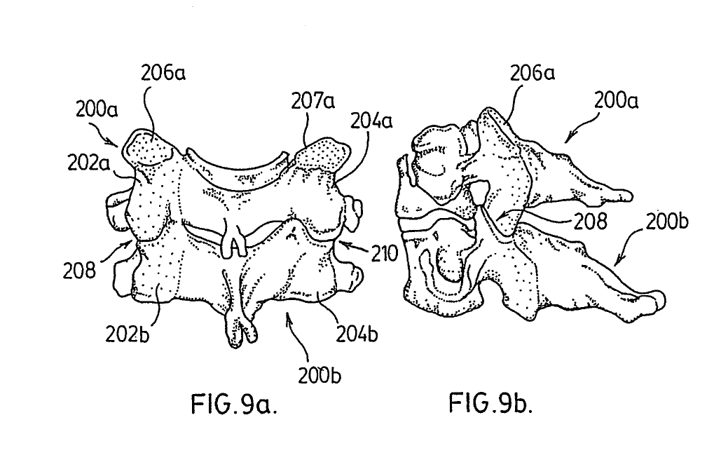

FIGS. 9A and 9B illustrate two adjacent vertebrae, a superior vertebra 200a and an inferior vertebra 200b. Each of superior and inferior vertebra includes, respectively, a right lateral mass (202a and 202b) and a left lateral mass (204a and 204b). FIG. 9A illustrates the right and left superior facets 206a and 207a, respectively, on the right and left lateral masses 202a and 204a. The opposing superior and inferior facets of the adjacent vertebrae form facet joints 208 and 210. As will be understood by persons skilled in the art, typical spinal structure would also include ligaments and the like (not shown in FIG. 9) to maintain the vertebrae in the normal position and to allow flexion there-between. As discussed above, in certain cases, such ligaments are rendered damaged or weakened (i.e. “insufficient”) for a variety of reasons. Such ligamentous insufficiency results in pain and/or damage to related spinal structures.

One method for reconstructing the ligaments of a facet joint involves the attachment of native, artificial, or synthetic ligamentous material so as to replace or augment ligaments within areas or regions of ligamentous insufficiency. It will be understood that several types of material are suitable for use as the ligamentous material of the present invention. The ligamentous material could be native or artificial ligament, tendon, or fascia, or manufactured material of a flexible (i.e. resilient) and durable nature. The ligament might also be a manufactured of a synthetic flexible matrix into which cells, such as fibroblasts, can impregnate or migrate. The matrix, by means of its structure and by chemicals possibly contained within it, could facilitate “directed growth”, such that the growth of the migrating cells within the matrix is encouraged. By including growth promoting agents within the matrix, the migrating cells deposit compounds, such as collagen and/or other proteins, so as to produce a new ligament made of human tissue. Generally, as used herein, the term “synthetic” may comprise both organic and non-organic material. For example, with respect to organic material, the “synthetic” ligament may comprise a ligamentous graft such as an autograft, allograft, or xenograft. Alternatively, the synthetic ligament may comprise other organic tissue having the required physical requirements such as fascia, or bovine pericardium. In general, the material is one that mimics the elastic nature of natural ligaments as found in the body. Ligaments serve to limit range of motion in a manner analogous to a tension band. In this capacity, ligaments found in the spine offer physiologic non-rigid spinal stabilization. With respect to inorganic materials for manufacturing the synthetic ligament, many options are possible. As will be appreciated by persons skilled in the art, the synthetic ligaments that can be used in the present invention are manufactured from a fabric or fabric-like tension band having physical properties approximate that of naturally occurring ligaments. By way of example only, one possible synthetic ligament that may be used in the implant described herein comprises the Leeds-Keio artificial ligament, which was developed by the University of Leeds (UK) and Keio University (Japan). Such artificial ligament comprises a polyester material having a mesh structure and has been investigated for use as a spinal ligament prosthesis (Suzuki K., Mochida J., Chiba M., Kikugawa H., Posterior Stabilization Of Degenerative Lumbar Spondylolisthesis With A Leeds-Keio Artificial Ligament. A Biomechanical Analysis In A Porcine Vertebral Model; Spine, 1999; 24 (1):26-31). Various other materials serving the same purpose will be known to persons skilled in the art.

The reconstruction of these regions of insufficiency allows for the maintenance of motion while reducing the loading of adjacent segments. By creating a lateral mass staple assembly as described herein, the facet joint can be reconstructed to allow motion but constraining flexion (i.e. forward or bending motion) so as to prevent overdistraction. In the present description the terms “staple” or “anchor plate” are used to describe an anchor that is secured to a bony structure. As discussed further below, such staple may be screwed, bolted, pinned or otherwise secured to bone. In one embodiment, the staples are screwed through an aperture provided therein. In general, the staples of the invention may be of any acceptable shape for the purpose described here. In one aspect, the staples are generally flat anchor plates. The staples may include one or more physical and/or chemical features to enhance bone, muscle, ligament and/or scar tissue in-growth so as to further secure the staple to the bone structure once implanted. The staples will generally be shaped, at least on their bone-contacting surface, to mate with the respective bone structure to which they are to be attached.

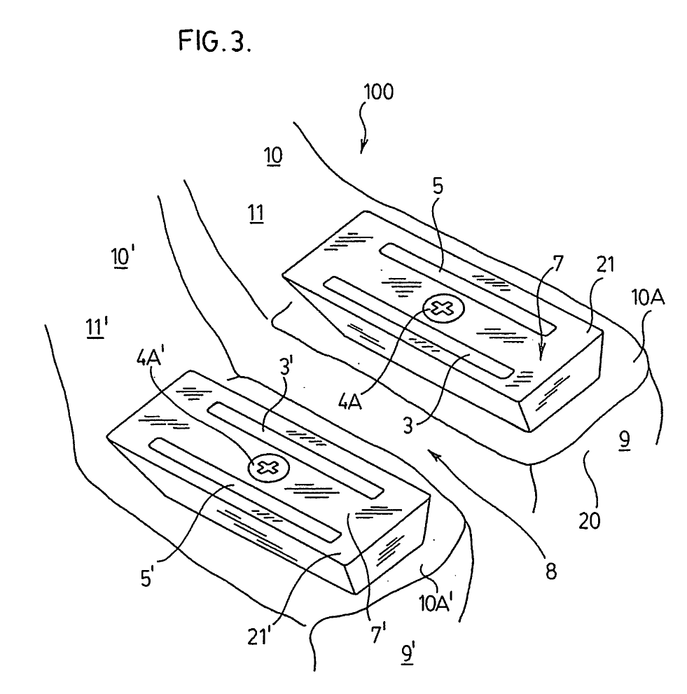

In FIGS. 3 to 5, there is shown a perspective view of a vertebral segment 100 having facets 10 and 10′ of vertebrae 10A and 10A′ and a facet joint 8, which make up the vertebral segment. As explained above, and as will be understood by persons skilled in the relevant art, facets are posterior structures of a vertebra which can articulate with facets of an adjacent vertebra to form facet joints that allows motion in the spinal column. Each vertebra has two (right and left) superior and two inferior facets. There is also shown, respectively, the lateral mass 9 and 9′ of vertebrae 10A and 10A′. It will be generally understood by persons skilled in the relevant art that the term “lateral mass” refers to the lateral expansion of the spinal ring such as of the cervical section of the spine, consisting of the facet joints and intervening bone as well as a tunnel through which the vertebral artery travels.

Also provided in FIGS. 3 to 5 is an embodiment of the lateral mass staple assembly 20 in accordance with the present invention. Lateral mass staple assembly 20 consists of two facet joint staples, namely a superior or cranial end staple 21 and an inferior or caudal end staple 21′. It will be understood that the terms “superior or cranial” and “inferior or caudal” refer to the vertical alignment of the staples when implanted. As shown in the embodiment depicted in the figures contained herein, the staples may comprise anchor plates, which are attached on superior and inferior vertebrae 10A and 10A′, respectively, by fasteners such as 4A. Fasteners 4A would generally include an anchoring means to engage the bone material of the lateral mass. In one embodiment, the fasteners include a screw portion, as shown in FIGS. 3 to 5, to serve as the anchoring means. In the figures contained herein, an embodiment is shown wherein one staple is anchored to each side of a vertebrae. However, it will be understood by persons skilled in the art, particularly based on the following description, that any number of staples may be used depending on the need. Thus, for example, two or more facet joint staples can be placed per side into the lateral masses.

The staples (i.e. anchor plates) of the present invention may be made of a suitable, surgical grade metal or metal alloy or other such durable material as will be known to persons skilled in the art.

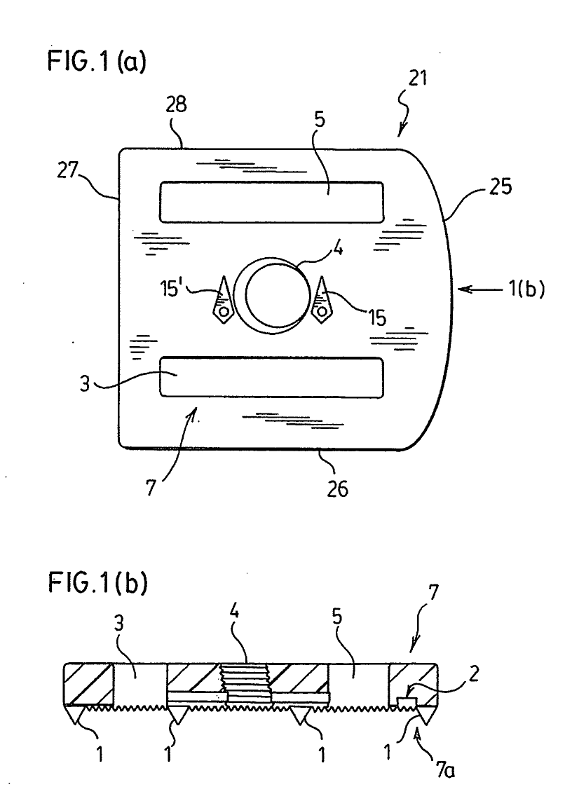

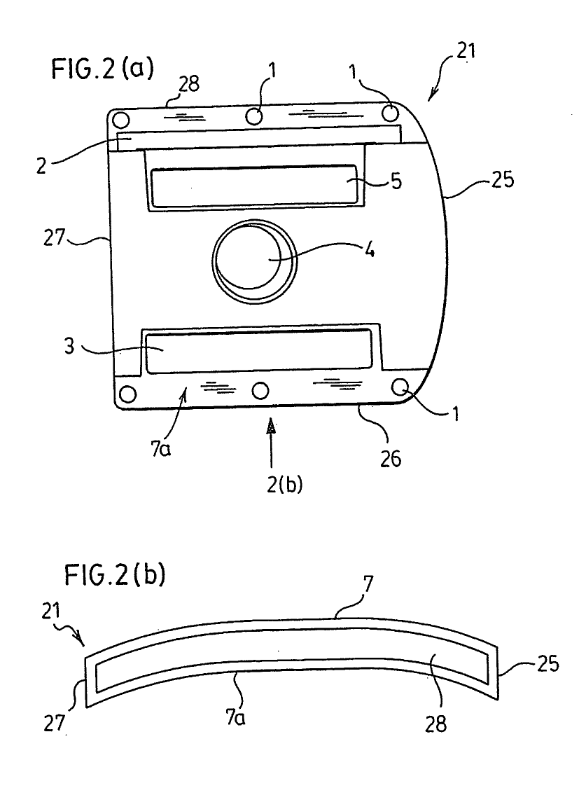

It will also be understood that, in a preferred embodiment, the facet joint staples are provided in left and right sided versions, which correspond to the left and right lateral aspects of a vertebra. As shown in the embodiment depicted in FIG. 1, each facet joint staple has a lateral side, which may have a curved contour 25 that allows for easy orientation. FIGS. 3 to 5 illustrate an alternate embodiment of the than that of FIG. 1 wherein a different staple design is shown. The lateral sided contour 25 (right side of diagram in FIGS. 1 and 2) abuts the lateral aspect of lateral mass 9 and 9′ and can be generally shaped to conform to the general shape of the lateral mass. This is particularly suitable for application on axis plates which have a curve to conform to the joint. The opposing side, namely medial side 27 (left side of diagram in FIGS. 1 and 2), has a generally straight portion that abuts the lamina 11 and 11′.

As shown in FIGS. 4 and 5, the invention is provided with a synthetic ligament 13, which is secured to facet joint staples 21 and 21′. As indicated above, the synthetic ligament 13 may be made from various materials as will be understood by persons skilled in the art.

FIGS. 1 and 2 show additional views of one embodiment of the facet joint staple of the present invention. The facet joint staple of the present invention may be manufactured in a variety of shapes and sizes to allow for use in different applications. A person skilled in the art will understand that the facet joint staple that will come in a variety of heights and widths to allow for use in different size patients as well as other vertebral segments. Considerations for the height and width of the facet joint staples can be (1) size of patient, (2) region of spine, i.e. cervical, thoracic, or lumbar, and (3) application, e.g. lateral mass or spinous process. In a preferred embodiment, the implant of the present invention can be approximately 2 to 3 mm thick. A facet joint staple having this thickness is preferred for attachment of ligaments to facet joints.

The facet joint staples 21 and 21′ include a first surface 7 and 7′, respectively which comprises the outer surface in the applied position. The staples also include a second, opposing surface comprising inner surface in the applied position, that is, the surface contacting the lateral mass or other spinal structure. In addition, the staples include first, second, third and fourth edges, 28, 25, 26 and 27 respectively. In the embodiment of the present invention shown in FIGS. 1 and 2, a first generally longitudinal aperture 3 is provided for each staple adjacent edge 26 and generally extends across the longitudinal axis extending from side 25 and side 27. Longitudinal aperture 3 also defines an opening between the outer and inner surfaces. Aperture 3 is adapted to receive a portion of ligament 13, as can be seen in FIGS. 3 to 5 and as will be described further below. A second longitudinal aperture 5 is also provided on each staple. Second aperture 5 also defines an opening extending between the outer and inner surfaces and is provided adjacent to edge 28. Similar in configuration to first aperture 3, second aperture 5 is adapted for receiving a portion of ligament 13.

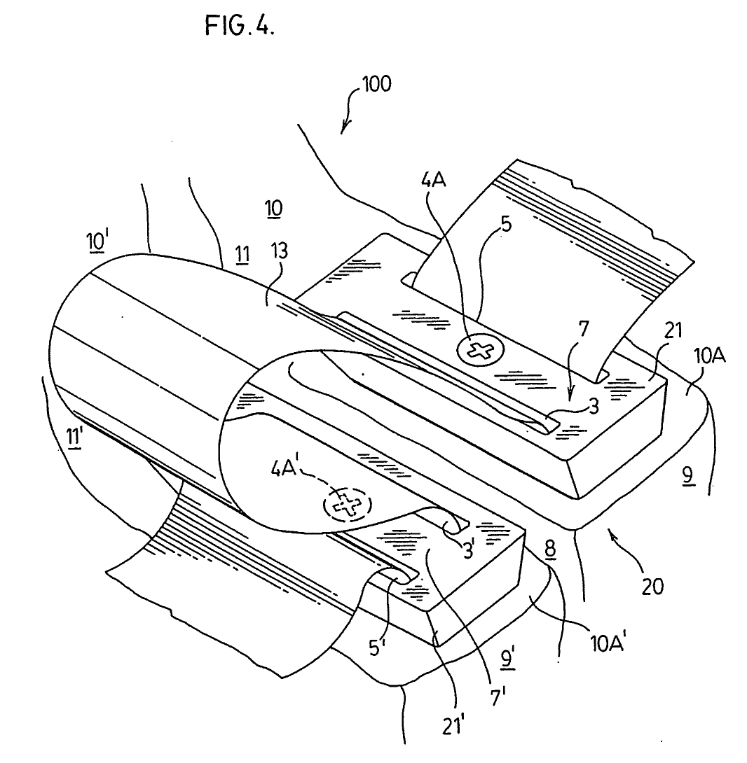

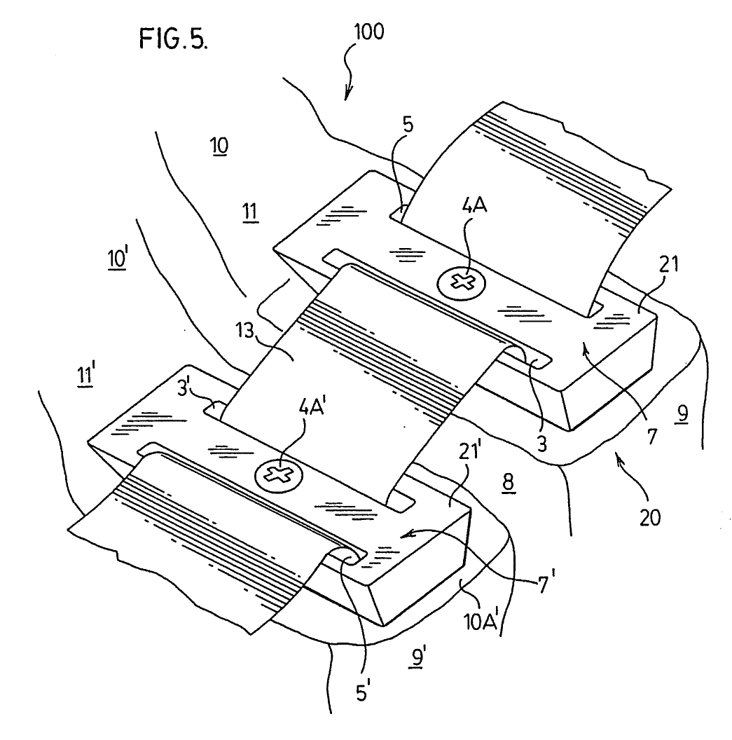

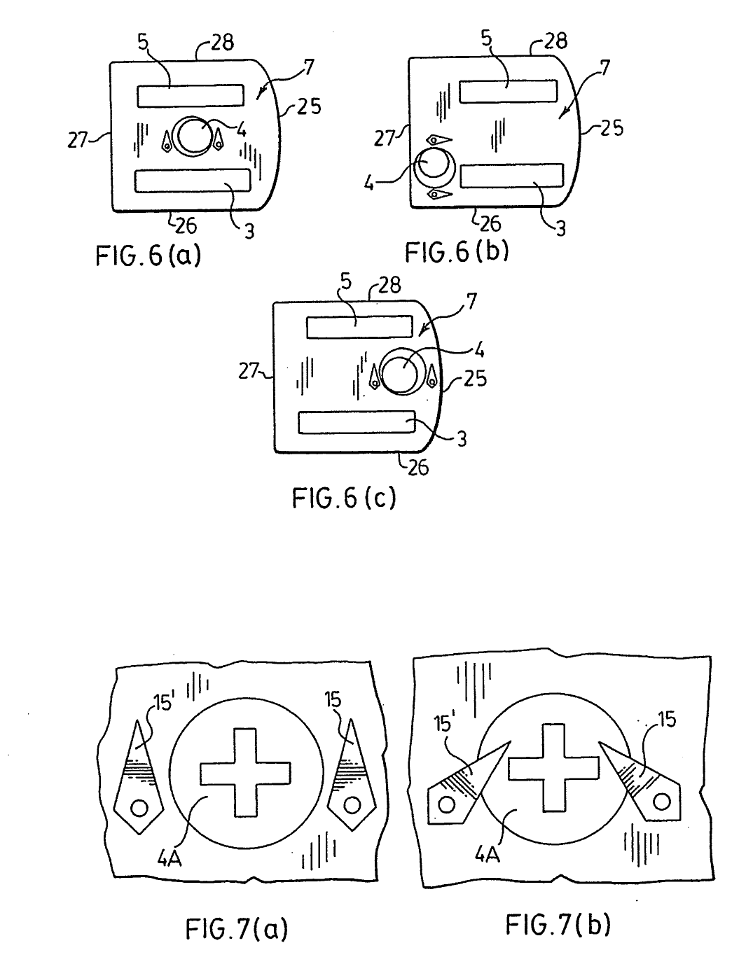

Each staple is further provided with a fastener-receiving aperture 4, extending through facet joint staple. In the embodiment of the present invention shown in FIGS. 1 and 2, fastener-receiving aperture 4 is provided generally in the center of facet joint staple 21. In alternate embodiments of the present invention, as seen in FIGS. 6(a), (b) and (c), the fastener-receiving aperture 4 may be in different locations about facet joint staple 21. Fastener-receiving aperture 4 is adapted to receive a fastener that will affix facet joint staple 21 to vertebrae 10A or 10A′. As shown in FIG. 2b, the facet joint staple may have a medial-lateral curve so as to generally conform with the surface of the vertebrae to which the facet joint staple is to be attached.

As shown in the figures, staple 21 is provided with apertures 3 and 5 while staple 21 is provided with equivalent apertures 3′ and 5′. Longitudinal apertures 3, 3′, 5 and 5′ are provided with generally smooth surfaces to as to allow ligament 13 to pass there-through. In a preferred embodiment, the ligament 13 is threaded through each of apertures 3 and 5 and 3′ and 5′, respectively as shown. In order to arrange lateral mass staple assembly 20 once facet joint staples 21 and 21′ have been placed on or affixed to vertebra 10A and 10A′, ligament 13 can be passed through these longitudinal apertures so as to provide the necessary stability to the joint as described herein.

As shown in the embodiment shown of FIGS. 4 and 5, in implanting the device of the invention, the synthetic ligament 13 is passed posterior-inferior through the apertures 3 and 5 and 3′ and 5′, respectively. As shown, in this manner, the ligament is oriented so as to lie between the spinal bone tissue and the inner surfaces 7 and 7′ respectively of staples 21 and 21′. As can be seen, in such orientation, the fasteners 4A and 4A′ used to anchor the staples will extend through the synthetic ligament 13. Staples 21 and 21′ can then be affixed through ligament 13 by fastener 4A.

FIGS. 4 and 5 show the embodiment of the present invention in use for the attachment of synthetic ligament 13 to a right facet joint. Two staples 21 and 21′ are shown, wherein one is placed on each side of the facet joint and wherein each staple is attached to the respective lateral mass by fasteners 4A and 4A′.

FIG. 4 shows the right facet joint in extension while FIG. 5 shows the right facet joint in flexion. As will be understood by persons skilled in the art, the term “extension” refers to an anterior to posterior motion of the spine (i.e. bending backward) whereas the term “flexion” refers to a posterior to anterior motion of the spine (i.e. bending forward). As seen in FIG. 5, when the spine in which the device of the invention is implanted is in flexion, the synthetic ligament 13 serves to limit the degree of flexion in a fashion akin to the in vivo facet joint capsule. On extension of the spinal segment, the lateral mass staple assembly of the invention does not limit the range of motion, with such limitation being the result of natural limits to extension, namely the facet joints abutting one another. As can be seen in FIG. 4, during extension, the synthetic ligament 13 can buckle and this built in laxity allows the subsequent normal movement of the facet joints in flexion.

As can be seen in FIG. 5, the synthetic ligament 13 has been stretched taut across the facet joint 8 in flexion thereby constraining the joint. The lateral mass staple assembly 20 therefore allows for the stabilization of the facet joint in flexion. As will be understood by persons skilled in the art, the stabilization implant described herein is particularly suited for implantation in the cervical segment of the spine so as to limit neck flexion. In particular, the device disclosed herein allows for reconstruction of the normal limitation to flexion provided by facet joint capsules in the cervical spine.

Finally, rotation movement (not shown) with lateral mass staple assembly 20 will be limited to a degree by the configuration of the underlying facet joint and contralateral facet joint. However, the properties of ligament 13 could limit excessive rotation, such as extremes of rotation to the point of subluxation limited by the capsule, as well as facet dislocation.

In a preferred embodiment, fastener-receiving aperture 4 can be threaded for receiving a fastener, such as a screw and more particularly such as a lateral mass screw as commonly known in the art. Examples of fasteners that may be used in conjunction with the facet joint staple of the present invention include screws, spikes, pins, rods, ties, or sutures. The fasteners can be inserted into the pars interarticularis, lateral mass, pedicles, spinous processes or any of the other elements in the bony spine. The fastener could also be inserted into artificial equivalents of the above. It will be understood, however, that the present invention is not limited to use with these fasteners. For example, in an alternate embodiment the fastener may be a bolt secured with a nut. Preferably, the fastener-receiving aperture 4 is angled, as shown in FIGS. 1 and 2 as well as FIGS. 6(a), (b) and (c), to allow for angular insertion of the fastener into adjacent bone, maximizing bone purchase and minimizing the chance of the fastener damaging other tissues. This angle is dependent upon the amount and position of underlying bone in various regions of the spine and the relationship of surrounding eloquent structures to the bone. The angle allows for a standard lateral mass screw to be used. Depending on the thickness of the plate portion of a staple (which may generally be approximately 2 mm or above), obtaining a suitable or satisfactory trajectory at angle through a straight hole could be a problem for the surgeon. The angle of fastener-receiving aperture 4 helps to overcome this problem, while allowing for variations in the thickness of the facet joint staple. In a preferred embodiment, the fastener-receiving aperture 4 can be angled between 20 to 40 degrees laterally (towards 25) from the outer (7, 7′) to the inner surface and 0 to 20 degrees superiorly (i.e. towards edge 28). More preferably in a cervical lateral mass and facet application, the angle of fastener-receiving aperture 4 can be 25° in both directions (up and down as well as medial and lateral, referred to as “upwards and outwards”) to allow for either lateral mass or pedicle fixation. It will be understood that the angulation and position of the fastener-receiving aperture can be varied to accommodate various types of fasteners, including pedicle or par screws.

The diameter of the fastener-receiving aperture may be varied depending on the diameter of the fastener used. As fastener 4A attaches or affixes facet joint staple to adjacent bone structures, it can also pass through ligament 13 so as to affix ligament 13. As such, the insert of the fastener 4A may aid the in-growth of bony-material around the ligament

In another embodiment, the outer surface 7, 7′ of the staples may be provided with a fastener lock for holding fastener 4A inserted into the fastener-receiving aperture 4 in place. In a preferred embodiment, as shown in FIG. 1(b), the lock consists of at least one rotatable flange 15 that can stop the movement of fastener 4A and prevent it from being removed from the fastener-receiving aperture 4. Rotatable flange 15 is provided on the first surface 7, 7′ adjacent to the fastener receiving aperture 4. Once fastener 4A has been used to affix the spinal implant to bone, the head of fastener 4A protrudes slightly above the first surface. Flange 15 can then be rotated over the head of fastener 4A, locking fastener 4A in place and preventing it from working free of the bone. As shown in FIG. 1 and in greater detail in FIG. 7, locking flanges 15 and 15′ can be moved from a first position which allows the entering and exiting of fastener 4A from the fastener-receiving aperture 4 to a second position that can stop the exiting of fastener 4A from the fastener-receiving aperture 4. Once fastener 4A has been inserted into fastener-receiving aperture 4, locking flanges 15 and 15′ can be moved from the first position to the second position to lock fastener 4A in place. Although this form of fastener-lock is preferred, the present invention is not limited to this fastener-lock. Various alternative fastener-locks which help to prevent the fastener from working free of the bone to which the implant is affixed could be substituted for the at least one flange.

FIGS. 6a, 6b, and 6c show various embodiments of the lateral mass staple of the invention in which the position of the fastener-receiving aperture 4 is varied. In a preferred embodiment, the positioning of the fastener-receiving aperture is based on the region of the spine where the implant is to be used. In FIG. 6a the fastener-receiving aperture is provided generally in the center of the facet joint staple. This embodiment is of particular use for the attachment of ligaments to the lateral mass of cervical vertebra. In this embodiment, the fastener-receiving aperture is preferably angled 25° upwards and outwards relative to the centre of the lateral mass. FIG. 6b shows an alternate embodiment of the facet joint staple in which the fastener-receiving aperture is provided adjacent to one end of the first longitudinal inferior aperture, such as aperture 3, and adjacent edges C and D. The arrangement of fastener-receiving aperture 4 shown in FIG. 6b is of particular use for the attachment of ligaments to the lamina of the C2 vertebra with C2 pars or C2 pedicle screws which can be placed through a more medially placed hole. The pedicle screw placement is also facilitated through a more lateral screw hole as shown. In this embodiment, fastener-receiving aperture 4 is preferably angled 45° superiorly.

FIG. 6c shows an alternate embodiment of the facet joint staple in which the fastener-receiving aperture 4 is provided adjacent the curved contoured edge B, and between the first aperture 3 and the second aperture 5. This embodiment is particularly useful for the attachment of ligaments to the C7 vertebra or the thoracic pedicle. In this embodiment, the fastener-receiving aperture is sized to accommodate larger screws, for example pedicle screws. The fastener-receiving aperture is angled 10° inferiorly (towards 26 from 7 to 7′) and 0 to 45° medially (towards 27 from 7 to 7′).

As shown in FIG. 2, each of the inside surfaces 7a and 7a′ of mass staple 21 or 21′, respectively, may also include at least one stabilizing member 1. As shown in FIG. 2, in one embodiment six or more stabilizing members are provided. Stabilizing members 1 can penetrate adjacent bony structures, thus allowing fixation of facet joint staples to the adjacent bony structures. Examples of stabilizing members include, but are not limited to, teeth, pins, and spikes. The at least one stabilizing member not only helps to attach the implant to adjacent structures, but also passes through the ligamentous material or ligament 13 to allow for bony ingrowth through the ligament. Each of the inside (i.e. bone contacting) surfaces 7 and 7′ of later mass staples 21 and 21′ preferably has a roughened, porous surface treatment or coating to allow for bony ingrowth which aids in the long term fixation of lateral mass staple assembly 20 to the lateral mass. In one embodiment the area of inside surfaces 7a and 7a′ between the first and second longitudinal apertures 3 and 5 can be rough to allow for increased bone growth in that area. In another embodiment, the inside surfaces 7a, 7a′ can be coated with a porous substance, such as titanium particle spray or plasmapore. In yet a further embodiment, inside surfaces 7a and 7a′ can include a hollow cage or similar mesh type structure as will be known to persons skilled in the art, in which to place a bone growth substance, such as bone morphogenic proteins (e.g. rhBMP2 or rhBMP-7) that stimulates bone growth into the cage, therefore incorporating bone into the facet joint staple. Various other similar treatments and coatings may also be provided with such features being apparent to persons skilled in the art.

As shown in FIGS. 1 and 2, inside surfaces 7a and 7a′ may also include one or more reservoirs 2. The reservoirs 2 may contain bone-fusion-enhancing materials, such as proteins that promote bone growth, in order to encourage in-growth of bone. In an embodiment of the present invention, reservoir 2 is a generally U-shaped indentation in surface 7′ along the surface furthest away from the facet joint. In other words, superior facet joint staple 21 would have reservoir 2 adjacent aperture 5 which is near the superior end, but for the inferior joint staple 21′, the reservoir would be located adjacent aperture 5′ which is near the inferior end of the structure. It will be understood that if three or more facet joint stables are to be used, then the middle staple or staples can have reservoirs adjacent both aperture 3 and aperture 5. The configuration in FIG. 3 is only an example of two abutting facet staples.

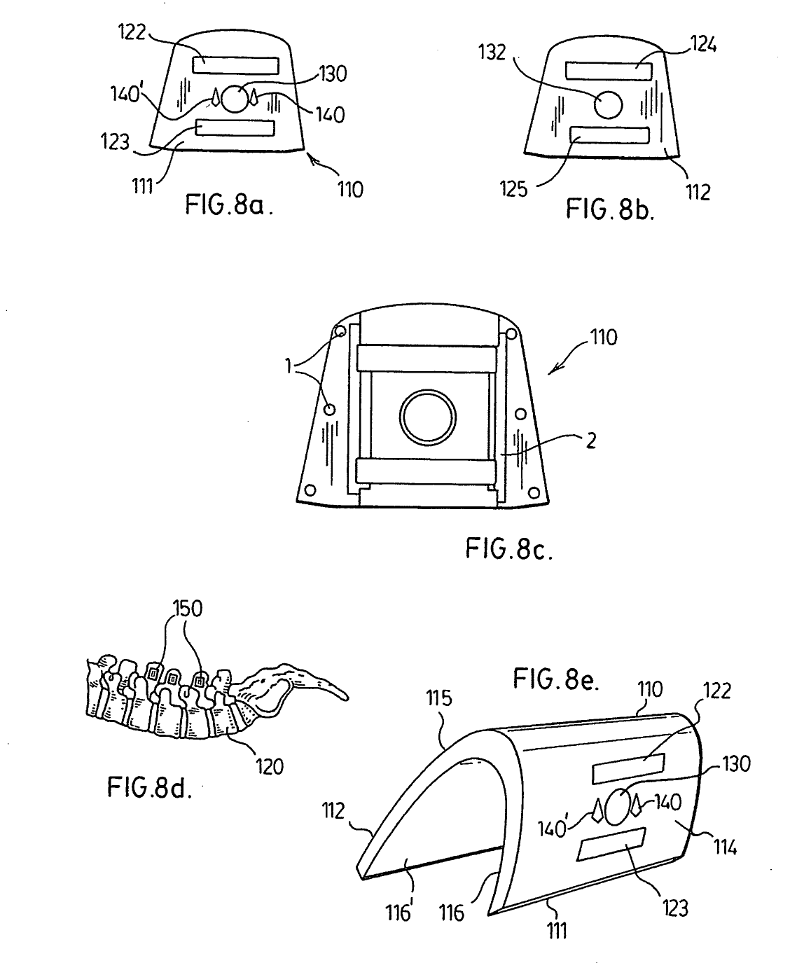

An alternate embodiment of the present invention is shown in FIGS. 8a to 8e wherein a staple is provided for attachment to a spinous process. Spinous process staple 110, as shown in FIG. 8(e), is designed to permit attachment of synthetic ligaments (not shown) to spinous processes 150 of FIG. 8(d) in the same manner as described above. Staple 110 as shown in FIGS. 8(a) to 8(e) may be used for ligamentous reconstruction of interspinous and supraspinous ligaments, and also for limiting flexion of the spine. Staple 110 is adapted to straddle spinous process 150 of vertebrae 120. Generally U-shaped staple 110 includes a first and second arm 111 and 112, respectively. As shown in FIG. 8(e), first and second arm have exterior surfaces 114 and 115 on one arm of the U-shaped staple 110, and an interior surfaces 116 and 116′ on the opposite side of the first surface 114 and second surfaces 115.

Each exterior surface, and its corresponding interior surface, includes at least two apertures (122,123,124,125) extending through the body of each arm to allow passage of ligaments therethrough. In addition, each exterior surface, and its corresponding interior surface, includes a fastener-receiving aperture (130, 132) to allow passage of a fastener therethrough and into the adjacent spinous process.

At least one of the exterior surfaces includes a fastener lock that functions as described further above. In the embodiment shown in FIGS. 8(a) to (e) the fastener lock is included on the first exterior surface 114 consists of a pair of flanges 140.

The first and second interior surfaces of the implant 110 may include all the features of the second surface 7′ of the staple 21 described above including stabilizing members, reservoirs for containing bony-fusion enhancing materials, and a plurality of pores to encourage in-growth of bone.

From the above discussion, various unique features of the invention can be determined. Firstly, the spinal stabilization implant discussed herein comprises an efficient facet joint capsule reconstruction, particularly for the cervical spine. It will also be understood that the embodiment described above for use on spinous processes also allows for ligamentous reconstruction of interspinous and supraspinous ligaments as well as allowing for dynamic limitation of flexion in the spine.

One of the unique features of the present device is that it provides for rapid and long term fixation of a synthetic ligament to lateral masses. This is achieved primarily by the structural features of the staples. For example, the porous surface structure of the staples promotes bony in-growth into to the staple. Further, the stabilizing members (for example pins) capture the bony regions of the lateral mass and, in addition, where they pass through the synthetic ligament, they promote bony in-growth there-through. The bony fusion enhancing material reservoirs (2) also promote bone in-growth through the synthetic ligament.

Another feature of the invention comprises the medial to lateral contouring of the staple undersurface which facilitates placement onto for example the lateral mass.

It will be understood by persons skilled in the art that various methods may be employed to secure the synthetic ligament to the staples. As described above, the synthetic ligament is, in one embodiment, held in place by both the securing fastener (e.g. a screw such as a lateral mass screw) and the stabilizing members (e.g. stabilizing pins). Alternatively, the synthetic ligament may be clipped, screwed or otherwise secured to the respective staple in any other manner while achieving the same purpose.

In the above description and as shown in FIGS. 1 to 8, an embodiment of the invention have been illustrated with respect to two staples being provided. However, as will be understood, in the course of stabilizing a spine in the present manner, it may be necessary to apply the present device to a number of vertebrae to achieve the desired stability. In this manner, the synthetic ligament can be continuous on each side, being secured to each staple along its length. Alternatively, the synthetic ligament can be provided in various sections, each section being secured in succession so as to effectively achieve a unified ligament. It will also be appreciated that the synthetic ligaments used in the present invention will be selected for length and elastic capability based on the specific needs.

The fastener receiving aperture of the staple is preferably angled, as explained above, to allow for, for example, the placement of lateral mass screws. In addition, this angle can be altered as needed in order to accommodate different screw trajectories such as screws into the pars of the C2 vertebra as well as pedicles. Various other angles and orientations will be apparent to persons skilled in the art depending upon the desired bone structure into which the staples are to be anchored. For example, the staples of the invention can be secured to artificial laminae, pedicles, lateral masses or vertebrae or any combination thereof.

As will be understood by persons skilled in the art, the straight medial edge of one embodiment of the staples will not interfere with potential decompressive procedures such as a laminectomy. As described above, the straight edge can straddle the of the decompression.

Another unique feature of the device described herein is the use of a “belt buckle” method of attaining immediate fixation of the synthetic ligament to the staple and the associated bone structure (i.e. lateral mass). Such method, along with the selection of a suitably elastic ligament material allows for a certain amount of elasticity similar to a normal facet joint capsule. This unique attachment means also stabilizes the facet in rotational motions as a result of it low profile (i.e. being located directly on the lateral mass).

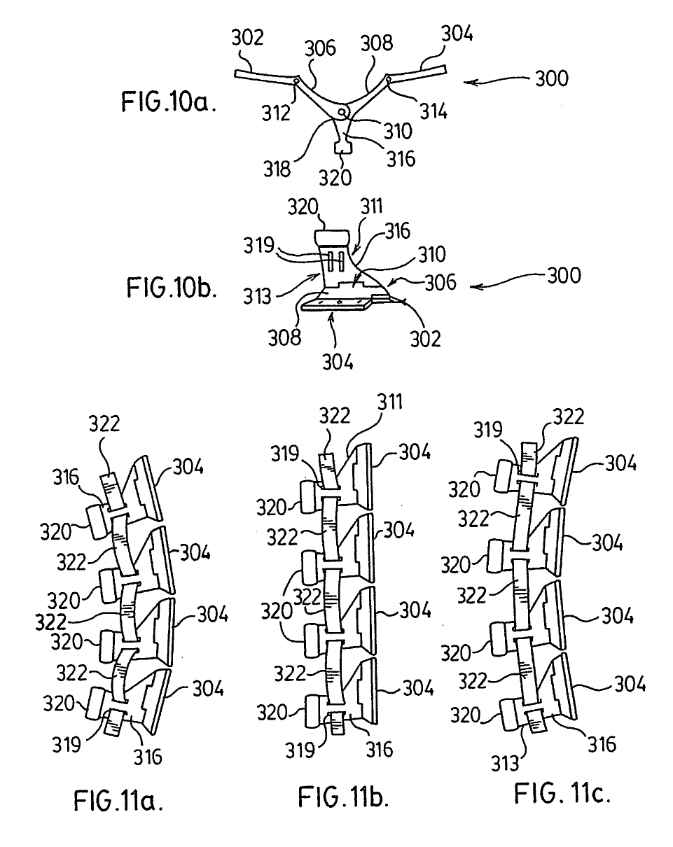

A further embodiment of the invention is shown in FIGS. 10 to 11. In these figures the stabilizing implant comprises an artificial spinous process and lamina for the vertebra with such implant being attached to other bony structures of the vertebra such as the lateral masses. In this embodiment, the implant 300 is designed to be positioned over a region of a spine where the naturally occurring spinous process and, in some case, lamina are excised to expose the spinal cord and dura. As will be known to persons skilled in the art, such a procedure may comprise a decompressive laminectomy. The implant 300 can be attached to various sections of the vertebra such as the lateral masses etc. Alternatively, the implant 300 can be attached to other staples such as those discussed above (and referred to as items 21 and 21′ in previous figures), or other similar prostheses such as an artificial facet joint and the like.

As shown in FIGS. 10a and 10b, the implant 300 includes two laterally extending and spaced apart staples 302 and 304, which, in one embodiment, comprise lateral mass staples. That is, the staples 302 and 304 are designed to be affixed to the two lateral masses on a vertebra. The staples 302 and 304 comprise anchor plates adapted to be attached to the desired bony structure. It will be appreciated that the staples 302 and 304 may include the various bony in-growth promoting means as described above. Further the staples 302 and 304 include a fastener receiving aperture to provide an aperture through which an anchoring means such as screws (i.e. lateral mass screws), pins and the like may be passed through to engage the underlying bony structure. As illustrated, the two staples 302 and 304 are generally flat plates each lying generally on the same plane. It will be understood that this description of orientation is not meant to be limiting in any way. That is, in many circumstances, the staples 302 and 304 may not be exactly co-planar and may, in fact, be slightly angled with respect to each other in order to adapt to the shape of the spinal segment.

According to one embodiment, extending from each of the staples 302 and 304 are spacer arms 306 and 308, respectively, which extend towards the other of the staples and such that each of the arms extend towards each other and meet at a junction 310. The junction 310 may comprise a moveable hinge. Alternatively, the junction 310 may be a fixed connection between the arms 306 and 308. As shown in FIG. 10b, the spacer arms may comprise plates. In one embodiment, the spacer arms 306, 308 extend away from the plane on which the staples 302, 304 lie so that the junction 310 comprises an apex point. The spacer arms 306 and 308 may be fixedly connected to the respective staple or may be connected with moveable hinges 312, 314, respectively. As can be seen in FIGS. 10a and 10b, the implant 300 assumes a “wing” like structure. In another embodiment, the staples themselves may be have an elongate structure thereby avoiding the need for the aforementioned spacers.

The implant further includes a fin 316 extending generally perpendicularly from the plane on which the staples 302, 304 lie. The fin 316 includes a first end 318 connected to the junction 310 and an opposite second end 320, preferably comprising a thickened portion. Such a thickened or bulbous structure provides increased surface area which facilitates attachment of scar tissue or artificial ligaments etc. Such a structure confers biomechanical advantage to the implant 300 by providing a “lever arm”, which helps in preventing unwanted flexion or kyphosis.

The first end 318 may be hingedly or fixedly connected to the junction 310. In one embodiment, the fin 316 may comprise an extension of one of spacer arms 306 or 308. It will also be understood that the spacer arms 306, 308 and the fin 316 may comprise one structure. As will be understood by persons skilled in the art, such a unitary structure may not allow for any movement between the respective parts. In another embodiment only the two spacer arms 306, 308 may comprise a single structure with the fin 316 and the staples 302 and 304 being independent structures. In yet another embodiment, the combination of the staples, spacer arms and fin may comprise a single structure.

In FIGS. 10 and 11, the staples 302 and 304 of the implant 300 are shown as being of roughly equal size. However, the size of each staple can be varied as needed. For example, in some cases, such as when a greater clearance of the dural sac is required on one side of the vertebra, a wider and/or longer staple may be required on such one side.

The fins include a superior edge 311 and an inferior edge 313, wherein such edges are in their superior/inferior positions when the implant is in place on an upright spine. As shown, in one embodiment, the inferior edge 313 is generally straight whereas the superior edge 311 includes a curve towards the inferior edge. Thus, when implanted, the anterior end of the fin 316 is wider than the posterior end. Further superior edge 311 includes a “swept back” shape.

As will be understood by persons skilled in the art and as discussed further with respect to FIGS. 11a to 11c, such a structure for the fin 316 (i.e. the combination of a straight inferior edge 313 and a “swept back” superior edge 311) minimizes or avoids any impediment to extension movements (i.e. either rostral or caudal movements) of the spine to occur without impediment. Namely, the tapered shape of the fin 316 prevents impact with adjacent fins or native bone structures during movement of the spine, particularly during extension movements. This feature is illustrated in FIGS. 11a to 11c. FIG. 11b illustrates a spine having a number of implants 300 when in the neutral state. In FIG. 11c, the spine is subjected to a flexion (i.e. rostral) movement. FIGS. 11a illustrates the implant containing spine in an extension (i.e. caudal) movement. As can be seen, in either case, the design of the fins 316 prevents contact between adjacent implants 300 or between the implants 300 and adjacent spinal structures.

The fins 316 are provided with one or more slots 319 or other such openings preferably extending generally longitudinally along the length thereof. Such slots or openings are similar in function to the apertures 3 and 5 discussed above in reference to previous embodiments of the invention. In one embodiment, at least two such slots are provided for reasons that will be apparent to persons skilled in the art in view of the present disclosure. However, as discussed further below, it will also be apparent that any number of slots may also be provided.

FIGS. 11a to 11c illustrate the implant 300 when implanted into a spine. The implants are secured to, for example, the lateral masses of vertebrae. In the illustration of FIGS. 11a to 11c, four such implants are shown and are vertically oriented with an upright spine. As shown, the implants are provided with a plurality of synthetic ligaments 322 connected to each fin 316 of the implants. The synthetic ligaments may be made from any suitable material as with the synthetic ligaments discussed above.

As illustrated in FIGS. 11a to 11c, a plurality of synthetic ligaments 322 with the terminal ends of each connecting the vertically adjacent implants 300 to each other. For example, in the embodiment illustrated in FIG. 11, the fins 316 are provided with two slots 319 separated vertically from each other. The slots are adapted to receive and retain one end of a ligament 322. Thus, as shown, a ligament 322 extends from the inferior slot of a superior implant 300 to the superior slot of the inferiorly adjacent implant. In this manner, each implant 300 is connected to the implants adjacent thereto. In situations where no adjacent implant is present (such as with the superior-most or inferior-most implants), a further synthetic ligament can be provided (where necessary) wherein such further ligament is secured at its terminal end (opposite to the implant) to naturally existing supporting ligaments. Alternatively, such terminal end can be attached to a lateral mass staple as discussed above (with respect to items 21 and 21′ of FIGS. 1 to 8).

The ends of the synthetic ligament 322 can be attached to the fins 316 by any acceptable method. For example, in one aspect, the ligaments may be sutured to the fins 316. In another aspect, the wing may be formed in two separable halves having there-between a toothed or pin structure which serves to engage one or more ends of the synthetic ligaments when the fin halves are secured together. In one aspect, the fins are designed to allow bony in-growth therein so as to seal the halves together and/or to further secure the synthetic ligament thereto.

In the above description, the synthetic ligament 322 was described as being provided by a plurality of segments each attached in succession to adjacent implants 300. However, it will be understood that the same effect can be provided by a continuous synthetic ligament, such continuous ligament being attached to each fin 316. The terminal ends of such continuous ligament may be secured to existing spinal elements as described above.

It will be appreciated that, in addition to promoting bony in-growth into the fin as mentioned above, various other sections (or the entire structure) of the implant 300 may be provided with various coatings, surface treatments, reservoirs etc containing structural or chemical factors to promote bone growth. Various examples of such factors were previously described. For example, various portions of the implant may be provided with a pitted surface to provide anchoring positions for bone, muscle, fascia, scar tissue and the like. Such surfaces may also be perforated with a plurality of holes to achieve the same purpose. Similarly, some or all surfaces of the implant can be coated with physical and/or chemical enhancers for promoting the growth of bone or other tissue (i.e. scar tissue, muscle etc.).

It will be understood that the range of motion between implants 300 will be dependent upon the length and elasticity of the synthetic ligaments. This is observed in comparing FIGS. 11a to 11c. Thus, it will be appreciated by persons skilled in the art that the degree of flexion (in particular) afforded by the implants 300 can be tailored as needed by choosing an appropriate length and type of material for the synthetic ligament. In another aspect, the synthetic ligament 322 of the implant 300 may be provided with one or more “stopper” mechanisms to limit the range of motion between the implants 300 and/or adjacent vertebrae. Limitations of this sort may be indicated when it is desired to modulate the progress of degenerative diseases. Such a “stopper” may comprise, for example, an extension to the ends 320 of the fins. In such case, the stoppers may be designed (sized and positioned) to interfere with each other during extension (FIG. 11a) so as to limit the range of the extension motion.

Although the invention has been described with reference to certain specific embodiments, various modifications thereof will be apparent to those skilled in the art without departing from the purpose and scope of the invention as outlined herein. The entire disclosures of all references recited above are incorporated herein by reference.

We claim:

1. A spinal stabilization implant for attaching to two adjacent vertebrae, said vertebrae having one or more bony structures, the implant comprising:
a first anchor plate for securing to a first of said vertebrae;
a second anchor plate for securing to a second of said vertebrae;
said first and second anchor plates including one or more fastener apertures for receiving fasteners to engage said bony structures of said vertebrae;
a resilient member extending between said first and second anchor plates allowing elastic relative movement between said anchor plates.

2. The implant of claim 1 wherein said first and second anchor plates are provided in pairs so as to straddle opposite sides of said vertebrae, wherein said implant comprises a pair of first anchor plates are securing to said first vertebra and a pair of second anchor plates for securing to said second vertebra.

3. The implant of claim 2 wherein said pairs of anchor plates are connected by a generally U shaped member.

4. The implant of claim 3 wherein said anchor plates include a bone contacting surface, said bone contacting surface including a means of engaging said vertebrae bony structure.

5. The implant of claim 4 wherein said means of engaging comprise a porous surface, stabilizing members, bone growth promoting factors or combinations thereof.

6. The implant of claim 5 wherein said one or more fastener apertures are provided angularly through said anchor plates.

7. The implant of claim 1 wherein said anchor plates include one or more slots extending there-through for receiving said resilient member.

8. The implant of claim 7 wherein said resilient member extends under the one or more fastener apertures along the bone contacting surface of said anchors.

9. The implant of claim 7 wherein said anchor plates include a means for engaging said resilient member.

10. The implant of claim 1 wherein the one or more fastener apertures are provided with a locking means to prevent removal of said fastener.

11. A spinal stabilization implant for attaching to two adjacent vertebrae, said vertebrae having one or more bony structures, the implant comprising:
a pair of first spaced apart anchor plates for securing to a first of said vertebrae;
a pair of second spaced apart anchor plates for securing to a second of said vertebrae;
each of said pairs of anchor plates generally being co-planar;
said first and second anchor plates including one or more fastener apertures for receiving fasteners to engage said bony structures of said vertebrae;
each of said pairs of anchor plates being connected to a generally planar fin, said fin being generally perpendicular to the plane containing the respective pairs of anchor plates and wherein said fin includes a first, anchor plate connecting end and an oppositely directed second, free end;
said fins being connected to a resilient member extending there-between.

12. The implant of claim 11 further comprising spacer arms extending between each of said pair of anchor plates and the respective fin thereby connecting said fin to said respective anchor plates.

13. The implant of claim 12 wherein said spacer arms are oppositely angularly disposed.

14. The implant of claim 11 wherein said fins are tapered wherein the length of said first end is longer than the second end.

15. The implant of claim 14 wherein said fin includes first and second edges extending between said first and second ends and wherein said first edge is straight and said second edge is angled thereby forming said taper.

16. The implant of claim 11 wherein said anchor plates include a bone contacting surface, said bone contacting surface including a means of engaging said vertebrae bony structure.

17. The implant of claim 16 wherein said means of engaging comprise a porous surface, stabilizing members, bone growth promoting factors or combinations thereof.

18. The implant of claim 17 wherein said one or more fastener apertures are provided angularly through said anchor plates.

19. The implant of claim 18 wherein said fins include at least one tissue growth promoting factors.

20. The implant of claim 19 wherein said factors comprise porous surfaces, pins, tissue growth promoting compounds or any combination thereof.
